(19) United States
(12) Patent Application Publication
(10) Pub. No.: US 2011/0071287 A1
(43) Pub. Date: Mar. 24, 2011

(54) PROCESS FOR THE PREPARATION OF EFAVIRENZ

(75) Inventors: Mukesh Kumar Madhra, Karnal (IN); Pankaj Kumar Singh, Kanpur (IN); Mukesh Sharma, Gurgaon (IN); Chandra Has Khanduri, Gurgaon (IN)

(73) Assignee: RANBAXY LABORATORIES LIMITED, New Delhi, Delhi (IN)

(21) Appl. No.: 12/990,578

(22) PCT Filed: May 1, 2009
(86) PCT No.: PCT/IB09/51796

§ 371 Date: Nov. 30, 2010

(30) Foreign Application Priority Data
May 1, 2008 (IN) 1108/DEL/2008

(51) Int. Cl. C07D 265/24

(52) U.S. Cl. 544/92

(57) ABSTRACT

The present invention relates to a process for the preparation of Efavirenz (Formula I), wherein triphosgene is used as a cyclizing agent.

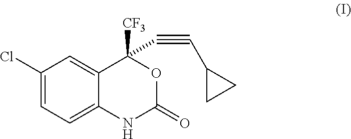

### FIELD OF THE INVENTION

The present invention relates to a process for the preparation of Efavirenz.

### BACKGROUND OF THE INVENTION

Efavirenz is chemically (S)-6-chloro-4-(cyclopropylethynyl)-1,4-dihydro-4-(trifluoromethyl)-2H-3,1-benzoxazin-2-one of Formula I.

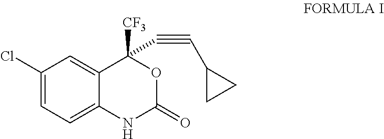

Efavirenz is a non-nucleoside, reverse transcriptase inhibitor and it is available in the market for the treatment of HIV-1 infection.

Efavirenz is prepared by cyclizing (2S)-2-(2-amino-5-chlorophenyl)-4-cyclopropyl-1,1,1-trifluorobut-3-yn-2-ol of Formula II.

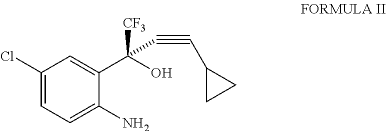

U.S. Pat. Nos. 6,040,480 and 6,028,237 describe a method for cyclizing the compound of Formula II by dissolving said compound in a mixture of heptanes and tetrahydrofuran and feeding phosgene to the solution at a temperature below 0° C. The method uses 80 mol of phosgene for cyclizing 54.3 mol of the compound of Formula II.

U.S. Pat. No. 6,015,926 describes a method for cyclizing the compound of Formula II by adding phosgene solution in toluene at 25° C. to a mixture of the compound of Formula II, toluene and aqueous potassium bicarbonate. The method uses 1.2 molar equivalents of phosgene for cyclizing 1 mol of the compound of Formula II. U.S. Pat. No. 6,015,926 also describes similar method wherein methyl t-butyl ether is used instead of toluene and phosgene is employed in the form of gas.

U.S. Pat. No. 5,922,864 describes methods for cyclizing the compound of Formula II using chloroformates such as 4-nitrophenyl chloroformate, methyl chloroformate and ethyl chloroformate. These methods use 1.05 to 2 molar equivalents of chloroformates for cyclizing 1 mol of the compound of Formula II.

U.S. Pat. No. 5,519,021 describes a method for cyclizing a racemic mixture of the compound of Formula II using 1,1′-carbonyldiimidazole. The method uses 0.259 mol of 1,1′-carbonyldiimidazole for cyclizing 0.0518 mol of a racemic mixture of the compound of Formula II.

The cyclization methods described in the prior art for preparing efavirenz employ toxic and hazardous cyclizing agent such as phosgene, which requires extremely careful handling. The present inventors have observed several problems associated with the generation, storage, usage and disposal of phosgene due to its toxicity and gaseous nature. The cyclization reaction using phosgene proceeds slowly and requires a long time for the completion of the reaction as phosgene has to be first absorbed in to the reaction medium. The cyclization methods involving chloroformates or 1,1′-carbonyldiimidazole as cyclizing agents have problems associated with the formation of by-products. For example, when 4-nitrophenyl chloroformate is used as a cyclizing agent, the efavirenz is formed along with p-nitrophenol as a major by-product, which is difficult to be removed. On the other hand, 1,1′-carbonyldiimidazole is sensitive to moisture and it also results in the formation of imidazole as a major by-product along with efavirenz. Thus, the methods involving chloroformates or 1,1′-carbonyldiimidazole as cyclizing agents require additional purification steps to obtain efavirenz with acceptable purity levels. Further, all the cyclization methods described in the prior art use excess quantities of the cyclizing agents, which impacts process economics in large scale operations. In case of chloroformates or 1,1′-carbonyldiimidazole, the excessive use of said cyclizing agents also increases the by-product formation.

### SUMMARY OF THE INVENTION

The present inventors have developed an advantageous process for the preparation of efavirenz, wherein triphosgene is used as a cyclizing agent. The present inventors have found that the use of triphosgene as a cyclizing agent for cyclizing the compound of Formula II tremendously minimizes the by-product formation and efavirenz can be obtained with high chemical and chiral purity without employing any additional purification steps. Since triphosgene is solid at room temperature, it avoids the handling problems associated with phosgene. The present process can be carried out using less than one molar equivalents of triphosgene. Thus, the present invention provides an efficient, cost effective and industrially applicable process for the preparation of efavirenz.

### DETAILED DESCRIPTION OF THE INVENTION

A first aspect of the present invention provides a process for the preparation of efavirenz of Formula I

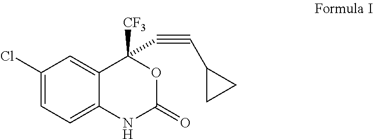

comprising cyclizing the compound of Formula II

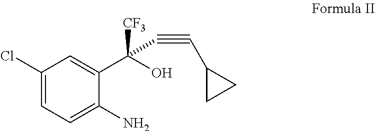

using triphosgene as a cyclizing agent.

The compound of Formula II may be prepared by the methods described in the prior art including those described in U.S. Pat. Nos. 6,028,237, 6,040,480 and 6,015,926. The cyclization reaction may be carried out in the presence of a solvent. The solvent may be selected from the group consisting of hydrocarbons, ethers, halogenated hydrocarbons, esters, nitriles, alcohols or mixtures thereof. The solvent may be, for example, hexane, heptane, toluene, methylene chloride, chloroform, methyl t-butyl ether, tetrahydrofuran or mixtures thereof. The reaction may be carried out in the presence of a base. The base may be an organic or inorganic base. The organic base may be an amine, for example, trialkyl amine, N-methylimidazole, quinuclidine, 1-methylpyrrolidine or morpholine. The inorganic base may be a hydroxide, for example, potassium, sodium, calcium, barium or magnesium hydroxide, or a carbonate, for example, sodium carbonate, potassium carbonate, magnesium carbonate, or a bicarbonate, for example, sodium bicarbonate or potassium bicarbonate.

Triphosgene may be used in less than about 1 molar equivalent to the molar quantity of the compound of Formula II. For example, about 0.3 to about 0.8 mol of triphosgene may be used for 1 mol of the compound of Formula II. The base and triphosgene may be added together or in optional order of succession to the compound of Formula II in the presence of the solvent. The addition of the base and triphosgene may be carried out at a temperature range of about −5° to about 35° C. Triphosgene may be added as a solid or as a solution in an organic solvent. The cyclization reaction may be facilitated by stirring the reaction mixture at a temperature range of about 0° C. to about 35° C., for example, about 5° to about 30° C. The reaction may be carried out for about 30 minutes to about 4 hours, for example, about 1 hour to about 2 hours. The efavirenz may be isolated from the reaction mixture by layer separation, concentration, distillation, filtration, decantation, precipitation or a combination thereof. The efavirenz may optionally be subjected to further recrystallization. The efavirenz so obtained has a chemical purity of about 99.7% or above, for example, about 99.9% or above, and a chiral purity of about 99.9% or above, for example, about 100%.

While the present invention has been described in terms of its specific embodiments, certain modifications and equivalents will be apparent to those skilled in the art and are intended to be included within the scope of the present invention.

### EXAMPLES

### Example 1

### Preparation of Efavirenz

Toluene (400 ml) was added to (2S)-2-(2-amino-5-chlorophenyl)-4-cyclopropyl-1,1,1-trifluorobut-3-yn-2-ol (100 g) at 20° to 25° C. and the reaction mixture was cooled to 10° C. Aqueous potassium bicarbonate solution (74.61 g in 300 ml of de-ionized water) was added to the reaction mixture in 10 to 15 minutes. Triphosgene (36.870 g pre-dissolved in 150 ml of toluene) was subsequently added to the reaction mixture in 45 to 75 minutes at 10° to 25° C. The reaction mixture was stirred at 10° to 25° C. for 60 minutes and the reaction mixture was quenched with methanol (7 ml) at 10° to 25° C. (All the above reaction steps were carried out under nitrogen atmosphere). The layers were separated and the organic layer was washed with de-ionized water followed by dilute hydrochloric acid. The solvent was recovered under reduced pressure and the solid obtained was re-crystallized with methanol: water (3:9) at 25° to 30° C. The solid was dried under reduced pressure at 85° to 90° C. for 15 to 18 hours to obtain the title compound.

Yield: 98.5 g

Chemical purity: 99.92%

Chiral purity: 100%

### Example 2

### Preparation of Efavirenz

Toluene (700 ml) was added to (2S)-2-(2-amino-5-chlorophenyl)-4-cyclopropyl-1,1,1-trifluorobut-3-yn-2-ol (100 gm) at 20° to 25° C. and the reaction mixture was cooled to 10° C. Triphosgene (36.870g) was added as a solid to the reaction mixture at 10° C. in 2 to 3 parts. Aqueous potassium bicarbonate solution (74.61 g in 300 ml of de-ionized water) was subsequently added to the reaction mixture in 45 to 75 minutes at 10° to 25° C. The reaction mixture was stirred at 10° to 25° C. for 60 minutes and the reaction mixture was quenched with methanol (7 ml) at 10° to 25° C. (All the above reaction steps were carried out under nitrogen atmosphere). The layers were separated and the organic layer was washed with de-ionized water followed by dilute hydrochloric acid. The solvent was recovered under reduced pressure and the solid obtained was re-crystallized with methanol: water (3:9) at 25° to 30° C. The solid was dried under reduced pressure at 85° to 90° C. for 15 to 18 hours to obtain the title compound.

Yield: 95 g

Chemical purity: 99.92%

Chiral purity: 100%

### Example 3

### Preparation of Efavirenz

Toluene (700 ml) was added to (2S)-2-(2-amino-5-chlorophenyl)-4-cyclopropyl-1,1,1-trifluorobut-3-yn-2-ol (100 g) at 20° to 25° C. and the reaction mixture was cooled to 0° C. Triphosgene (36.870 g) was added as a solid to the reaction mixture at 0° to 5° C. in 2 to 3 parts. Aqueous potassium bicarbonate solution (74.61 g in 300 ml of de-ionized water) was subsequently added to the reaction mixture in 45 to 75 minutes at 5° to 10° C. The reaction mixture was stirred at 5° to 10° C. for 60 minutes and the reaction mixture was quenched with methanol (7 ml) at 10° to 25° C. (All the above reaction steps were carried out under nitrogen atmosphere). The layers were separated and the organic layer was washed with de-ionized water followed by dilute hydrochloric acid. The solvent was recovered under reduced pressure. The solid obtained was re-crystallized with methanol: water (3:9) at 25° to 30° C. The solid was dried under reduced pressure at 85° to 90° C. for 15 to 18 hours to obtain the title compound.

Yield: 96 g

Chemical purity: 99.83%

Chiral purity: 100%

### Example 4

### Preparation of Efavirenz

Toluene (700 ml) was added to (2S)-2-(2-amino-5-chlorophenyl)-4-cyclopropyl-1,1,1-trifluorobut-3-yn-2-ol (100 g) at 20° to 25° C. Triphosgene (36.870 g) was added as a solid to the reaction mixture at 20° to 25° C. in 2 to 3 parts. Aqueous potassium bicarbonate solution (74.61 g in 300 ml of de-ionized water) was added to the reaction mixture in 45 to 75 minutes at 25° to 30° C. The reaction mixture was stirred at 25° to 30° C. for 60 minutes and the reaction mixture was quenched with methanol (7 ml) at 10° to 25° C. (All the above reaction steps were carried out under nitrogen atmosphere). The layers were separated and the organic layer was washed with de-ionized water followed by dilute hydrochloric acid. The solvent was recovered under reduced pressure. The solid obtained was re-crystallized with methanol: water (3:9) at 25° to 30° C. The solid was dried under reduced pressure at 85° to 90° C. for 15 to 18 hours to obtain the title compound.

Yield: 96 g

Chemical purity: 99.72%

Chiral purity: 99.90%

### Example 5

### Preparation of Efavirenz

Dichloromethane (800 ml) was added to (2S)-2-(2-amino-5-chlorophenyl)-4-cyclopropyl-1,1,1-trifluorobut-3-yn-2-ol (100 g) at 20° to 25° C. and the reaction mixture was cooled to 10° C. Aqueous potassium bicarbonate solution (74.61 g in 300 ml of de-ionized water) was added to the reaction mixture in 10 to 15 minutes. Triphosgene (36.870 g pre-dissolved in 150 ml of dichloromethane) was added in 45 to 75 minutes at 10° to 25° C. The reaction mixture was stirred at 10° to 25° C. for 60 minutes and the reaction mixture was quenched with methanol (7 ml) at 10° to 25° C. (All the above reaction steps were carried out under nitrogen atmosphere). The layers were separated and the organic layer was washed with de-ionized water followed by dilute hydrochloric acid. The solvent was recovered under reduced pressure. The solid obtained was re-crystallized with methanol: water (3:9) at 25° to 30° C. The solid was dried under reduced pressure at 85° to 90° C. for 15 to 18 hours to obtain the title compound.

Yield: 96.2 g

Chemical purity: 99.89%

Chiral purity: 100%

### Example 6

### Preparation of Efavirenz

Methyl t-butyl ether (900 ml) was added to (2S)-2-(2-amino-5-chlorophenyl)-4-cyclopropyl-1,1,1-trifluorobut-3-yn-2-ol (100 gm) at 20° to 25° C. and reaction mixture was cooled to 10° C. Aqueous potassium bicarbonate solution (74.61 g in 300 ml of de-ionized water) was added in 10 to 15 minutes. Triphosgene (36.870 g pre-dissolved in 150 ml of Methyl t-butyl ether) was added in 45 to 75 minutes at 10° to 25° C. The reaction mixture was stirred at 10° to 25° C. for 60 minutes and the reaction mixture was quenched with methanol (7 ml) at 10° to 25° C. (All the above reaction steps were carried out under nitrogen atmosphere). The layers were separated and the organic layer was washed with de-ionized water followed by dilute hydrochloric acid. The solvent was recovered under reduced pressure. The solid obtained was re-crystallized with methanol: water (3:9) at 25° to 30° C. The solid was dried under reduced pressure at 85° to 90° C. for 15 to 18 hours to obtain the title compound.

Yield: 97.1 g

Chemical purity: 99.91%

Chiral purity: 100%

We claim:

1. A process for the preparation of efavirenz of Formula I 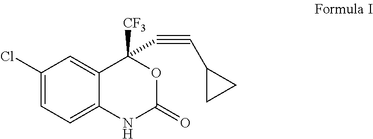
comprising cyclizing a compound of Formula II 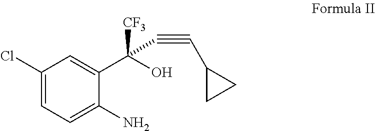
using triphosgene as a cyclizing agent.

2. A process according to claim 1, wherein the cyclization is carried out in the presence of a solvent.

3. A process according to claim 2, wherein the solvent is selected from the group consisting of hydrocarbons, ethers, halogenated hydrocarbons, esters, nitriles, alcohols or mixtures thereof.

4. A process according to claim 1, wherein the cyclization is carried out in the presence of a base.

5. A process according to claim 1, wherein triphosgene is used in less than about 1 molar equivalent to the molar quantity of the compound of Formula II.

6. A process according to claim 1, wherein the cyclization reaction is facilitated by stirring the reaction mixture.

7. A process according to claim 6, wherein the stirring is carried out at a temperature range of about 0° C. to about 35° C.

8. A process according to claim 6, wherein the stirring is carried out for about 30 minutes to about 4 h.

9. Efavirenz prepared according to the process of claim 1 having a chemical purity of at least 99.7% o and a chiral purity of at least 99.9%.

10. Efavirenz prepared according to the process of claim 1 having a chemical purity of at least 99.9% and a chiral purity of 100%.
